(19) United States
(12) Patent Application Publication
(10) Pub. No.: US 2023/0398075 A1
(43) Pub. Date: Dec. 14, 2023

(54) NANOPARTICLE SYNTHESIS SYSTEMS AND METHODS EMPLOYING VORTEX FLOW FOCUSING

(71) Applicant: UNIVERSITY OF MARYLAND, COLLEGE PARK, College Park, MD (US)

(72) Inventors: Jung Yeon HAN, North Bethesda, MD (US); Donald Lad DEVOE, Bethesda, MD (US)

(21) Appl. No.: 18/208,507

(22) Filed: Jun. 12, 2023

Related U.S. Application Data
(60) Provisional application No. 63/377,178, filed on Sep. 26, 2022.
(60) Provisional application No. 63/366,240, filed on Jun. 10, 2022.

(51) Int. Cl. A61K 9/127; B01J 13/08; B01F 33/3011; B01F 25/10

(52) U.S. Cl. CPC A61K 9/1277; B01J 13/08; B01F 33/3011; B01F 25/10; B01F 2025/913

(57) ABSTRACT

A first inlet flow can be directed along an axial direction in a hydrocyclonic flow cell. The first inlet flow can include first constituent molecules. At a same time, one or more second inlet flows can be directed along a circumferential direction of the hydrocyclonic flow cell. Each second inlet flow can include a buffer solution. The first inlet flow can be subjected to flow focusing by a surrounding primary vortex formed by the one or more second inlet flows, so as to generate a flow comprising a plurality of nanoparticles at an outlet of the hydrocyclonic flow cell. Each nanoparticle can be formed by a respective plurality of the first constituent molecules.

### CROSS-REFERENCE TO RELATED APPLICATIONS

The present application claims the benefit of U.S. Provisional Application No. 63/366,240, filed Jun. 10, 2022, entitled “Microfluidic Vortex Focusing for High-Throughput Synthesis of Size-Tunable Liposomes,” and U.S. Provisional Application No. 63/377,178, filed Sep. 26, 2022, entitled “Microfluidic Vortex Focusing for High-Throughput Synthesis of Size-Tunable Liposomes,” each of which is hereby incorporated by reference herein in its entirety.

### STATEMENT REGARDING FEDERALLY SPONSORED RESEARCH

This invention was made with government support under R21HL159590 awarded by the National Institutes of Health (NIH), and under CMMI1562468 and CMMI1950234 awarded by the National Science Foundation (NSF). The government has certain rights in the invention.

### FIELD

The present disclosure relates generally to particle synthesis, and more particularly, to nanoparticle synthesis via vortex flow focusing.

### BACKGROUND

The use of liposomes and other lipid-based nanoparticles as nanoscale drug carriers for the controlled delivery of therapeutic agents has been harnessed for applications in cancer, infectious diseases, immune modulation, vaccine delivery, and beyond. Liposomal nanoparticles support the encapsulation of the full range of hydrophilic, amphipathic, and lipophilic drug compounds within unilamellar lipid vesicles, which protect the loaded drugs from degradation by the mononuclear phagocytic system (MPS) or endogenous enzymes. Liposome properties may also be modified during or after vesicle formation to engineer desirable biodistribution profiles via targeted cell delivery, thereby addressing issues of poor bioavailability, low plasma solubility, non-specific targeting, and high clearance rate often associated with free drug agents.

The efficacy and toxicity of liposome-based drug delivery systems can be influenced by liposome size. Smaller vesicles can exhibit more uniform pharmacodynamic characteristics and offer improved bioavailability through the enhanced permeability and retention (EPR) effect, which can allow smaller liposomal nanoparticles to exhibit increased accumulation within tumors due to higher vascular permeability within these tissues. In addition, liposomes below approximately 100 nm can pass the blood-brain barrier, while vesicles in the 30-40 nm range can enhance transdermal transport. Nanoparticle size can also affect blood circulation time, biodistribution, cell uptake, subcellular localization, and targeting efficiency. Size-dependent liposomal drug toxicity has also been reported, with higher toxicity resulting from larger liposomes due to their increased retention in healthy tissues. Thus, tuning nanoparticle size to a desired range while maintaining low polydispersity can help optimize, or at least improve, nanomedicine performance.

Continuous-flow microfluidic techniques have been explored to provide control over the microenvironment during lipid self-assembly. For example, in microfluidic flow focusing, lipids dissolved in a water-miscible polar solvent are injected into a microfluidic junction with aqueous buffer sheathing the lipid solution and hydrodynamically focusing the lipid solution into a narrow sheet. Diffusive transport of solvent and water in the laminar flow environment rapidly reduce lipid solubility during focusing to promote vesicle self-assembly. Due to the small lateral length scales of the focused lipid stream, smaller liposomes with decreased polydispersity can be achieved using this technique. Another microfluidic liposome synthesis method employs rapid mixing to achieve a rapid change in solubility and small diffusive length scales through increased interfacial area in a binary fluid system. Rapid mixing is achieved using periodic microstructures, such as a herringbone pattern, baffles, or toroidal or twisted microfluidic channels to generate localized chaotic advection at high flow velocity. Although microfluidic mixers can be simpler to operate than flow focusing devices, the resulting liposome populations tend to exhibit higher size variance and a more limited size range.

While the continuous-flow nature of these microfluidic techniques eliminates the need for multiple handling steps associated with batch methods, the small microchannel dimensions and laminar flow requirements constrain the throughput of the technology. Various modified flow focusing and micromixer designs have been developed to address this limitation, but with reduced size controllability and higher observed polydispersity due to the larger geometries required to support the increased buffer and lipid flow rates. Scaling these microfluidic platforms for high production throughput without sacrificing size control has thus proven to be challenging. Embodiments of the disclosed subject matter may address one or more of the above-noted problems and disadvantages, among other things.

### SUMMARY

Embodiments of the disclosed subject matter provide systems and methods for forming nanoparticles using vortex flow focusing. In some embodiments, a tangentially-directed flow in a microfluidic flow cell generates a primary vortex surrounding an axially-directed flow with constituent molecules for forming desired nanoparticles. The primary vortex subjects the axially-directed flow to simultaneous flow focusing and mixing, thereby allowing the formation of nanoparticles. In some embodiments, the constituent molecules can comprise inorganic molecules or organic molecules. In some embodiments, the sizes of the formed nanoparticles in the flow cell can be controlled by adjusting the respective flow rates of the tangentially-directed and axially-directed flows.

In one or more embodiments, a method for forming a plurality of nanoparticles can comprise (a) directing a first inlet flow along an axial direction in a hydrocyclonic flow cell. The first inlet flow can comprise first constituent molecules. The method can further comprise (b) at a same time as (a), directing one or more second inlet flows along a circumferential direction of the hydrocyclonic flow cell. Each second inlet flow can comprise a buffer solution. The directing of (a) and (b) can be such that the first inlet flow is subjected to flow focusing by a surrounding primary vortex formed by the one or more second inlet flows, so as to generate a flow comprising the plurality of nanoparticles at an outlet of the hydrocyclonic flow cell. Each nanoparticle can be formed by a respective plurality of the first constituent molecules.

In one or more embodiments, a nanoparticle synthesis system can comprise a hydrocyclonic flow cell, which can comprise a cylindrical section, an inlet nozzle, and a conical section. The cylindrical section can comprise a circumferentially-extending wall with one or more tangentially-oriented inlet ports. The inlet nozzle can be surrounded by the circumferentially-extending wall of the cylindrical section and can provide an axially-oriented inlet port. The conical section can extend from an axial end of the circumferentially-extending wall of the cylindrical section toward an axially-oriented outlet port. The conical section can be tapered along at least a portion of its length along an axial direction. The axially-oriented inlet port and the axially-oriented outlet port can be substantially co-axial. A radially-outer surface of the inlet nozzle can be tapered along at least a portion of its length along the axial direction.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Embodiments will hereinafter be described with reference to the accompanying drawings, which have not necessarily been drawn to scale. Where applicable, some elements may be simplified or otherwise not illustrated in order to assist in the illustration and description of underlying features. Throughout the figures, like reference numerals denote like elements.

FIG. 1A is a simplified schematic diagram illustrating aspects of forming a plurality of nanoparticles via vortex flow focusing, according to one or more embodiments of the disclosed subject matter.

FIGS. 1B-1E are simplified schematic diagrams of nanoparticle synthesis systems employing vortex flow focusing, according to one or more embodiments of the disclosed subject matter.

FIG. 2 depicts a generalized example of a computing environment in which the disclosed technologies may be implemented.

FIG. 3A shows a cutaway perspective view of a hydrocyclonic flow cell, according to one or more embodiments of the disclosed subject matter. FIG. 3B is a plan view of the cylindrical portion of the hydrocyclonic flow cell of FIG. 3A.

FIG. 3C is a transparent perspective view of a nanoparticle synthesis system with a hydrocyclonic flow cell, according to one or more embodiments of the disclosed subject matter.

FIG. 3D is a close-up sectional view illustrating vortex formation around an inlet nozzle in the hydrocyclonic flow cell of the nanoparticle synthesis system of FIG. 3C.

FIG. 3E is a plane view of the cylindrical portion of another hydrocyclonic flow cell employing multiple tangential inlets, according to one or more embodiments of the disclosed subject matter.

FIG. 4A shows a cutaway perspective view of another hydrocyclonic flow cell with a pair of secondary inlet ports, according to one or more embodiments of the disclosed subject matter. FIG. 4B is a plan view of the cylindrical portion of the hydrocyclonic flow cell of FIG. 4A.

FIG. 5A shows a cutaway perspective view of another hydrocyclonic flow cell with an annular secondary inlet port, according to one or more embodiments of the disclosed subject matter. FIG. 5B is a plan view of the cylindrical portion of the hydrocyclonic flow cell of FIG. 5A.

FIG. 6A illustrates an experimental configuration for hydrodynamic flow focusing at a total flow rate of 60 mL/min and flow rate ratio of ethanol/lipid solution to water of 1:30.

FIG. 6B shows simulated flow streamlines (left) and ethanol concentration profile (right) for the experimental configuration of FIG. 6A.

FIGS. 6C-6D are images and graphs, respectively, of radial solvent profiles at different axial distances from the lipid injection point in the experimental configuration of FIG. 6A.

FIG. 7A illustrates an experimental configuration for vortex flow focusing at a total flow rate of 60 mL/min and flow rate ratio of ethanol/lipid solution to water of 1:30.

FIG. 7B shows simulated flow streamlines (left) and ethanol concentration profile (right) for the experimental configuration of FIG. 7A.

FIGS. 7C-7D are images and graphs, respectively, of radial solvent profiles at different axial distances from the lipid injection point in the experimental configuration of FIG. 7A.

FIG. 8A is an image of a fabricated nanoparticle synthesis system employing a hydrocyclonic flow cell.

FIG. 8B is a contrast-enhanced micro-computed tomographic (CT) image of the hydrocyclonic flow cell of FIG. 8A.

FIG. 8C is a close-up view of an inlet region of the hydrocyclonic flow cell of FIG. 8B.

FIG. 9A is a graph of average liposome diameter versus flow rate ratio (buffer:lipid) using the nanoparticle synthesis system of FIG. 8A, for a lipid mixture of DMPC:cholesterol:DCP (5:4:1 molar ratio), a total flow rate of 60 mL/min, and an initial lipid concentration of 10 mM.

FIG. 9B is a graph of polydispersity index versus flow rate ratio (buffer:lipid) using the nanoparticle synthesis system of FIG. 8A, for a lipid mixture of DMPC:cholesterol:DCP (5:4:1 molar ratio), a total flow rate of 60 mL/min, and an initial lipid concentration of 10 mM.

FIG. 9C shows normalized size distribution plots for liposomes formed using the nanoparticle synthesis system of FIG. 8A at different flow rate ratios (lipid:buffer).

FIG. 10A is a graph of average liposome diameter versus total flow rate using the nanoparticle synthesis system of FIG. 8A, for a lipid mixture of DMPC:cholesterol:DCP (5:4:1 molar ratio), a fixed flow rate ratio of 50 (buffer:lipid), and a total lipid concentration of either 10 mM or 20 mM.

FIG. 10B is a graph of polydispersity index versus total flow rate using the nanoparticle synthesis system of FIG. 8A, for a lipid mixture of DMPC:cholesterol:DCP (5:4:1 molar ratio), a fixed flow rate ratio of 50 (buffer:lipid), and a total lipid concentration of either 10 mM or 20 mM.

FIG. 10C shows normalized size distribution plots for liposomes formed using the nanoparticle synthesis system of FIG. 8A at different total flow rates and a total lipid concentration of 10 mM.

FIG. 10D shows normalized size distribution plots for liposomes formed using the nanoparticle synthesis system of FIG. 8A at different total flow rates and a total lipid concentration of 20 mM.

FIG. 11 is a histogram of diameters for liposomes formed using the nanoparticles synthesis system of FIG. 8A for a lipid mixture of DMPC:cholesterol:DCP (5:4:1 molar ratio), a total flow rate of 80 mL/min, a flow rate ratio of 50 (buffer:lipid), and a total lipid concentration of 10 mM.

FIG. 12A is a graph of average liposome diameter versus total flow rate using the nanoparticle synthesis system of FIG. 8A, for a lipid mixture of either DMPC:cholesterol:DCP (5:4:1 molar ratio) or DMPC:cholesterol:PEG2k-PE (5:4:1 molar ratio), a fixed flow rate ratio of 50 (buffer:lipid), and a total lipid concentration of 10 mM.

FIG. 12B is a graph of polydispersity index versus total flow rate using the nanoparticle synthesis system of FIG. 8A, for a lipid mixture of either DMPC:cholesterol:DCP (5:4:1 molar ratio) or DMPC:cholesterol:PEG2k-PE (5:4:1 molar ratio), a fixed flow rate ratio of 50 (buffer:lipid), and a total lipid concentration of 10 mM.

FIG. 12C shows normalized size distribution plots for liposomes formed from DMPC:cholesterol:PEG2k-PE (5:4:1 molar ratio) using the nanoparticle synthesis system of FIG. 8A at different total flow rates and a total lipid concentration of 10 mM.

FIG. 13A is a graph of average liposome diameter versus flow rate ratio (buffer:lipid) using the nanoparticle synthesis system of FIG. 8A, for a total flow rate ratio of 80 mL/min and either a lipid mixture of either DMPC:cholesterol:DCP (5:4:1 molar ratio) at 30 mM total lipid concentration or DMPC:cholesterol:PEG2k-PE (5:4:1 molar ratio) at a total lipid concentration of either 40 mM or 60 mM.

FIG. 13B is a graph of polydispersity index flow rate ratio (buffer:lipid) using the nanoparticle synthesis system of FIG. 8A, for a total flow rate ratio of 80 mL/min and either a lipid mixture of either DMPC:cholesterol:DCP (5:4:1 molar ratio) at 30 mM total lipid concentration or DMPC:cholesterol:PEG2k-PE (5:4:1 molar ratio) at a total lipid concentration of either 40 mM or 60 mM.

FIG. 13C compares liposome production rate results for a vertical flow focusing (VFF) system, a 3D micromixing system, and the nanoparticle synthesis system of FIG. 8A for a flow rate ratio of 10 and either a lipid mixture of either DMPC:cholesterol:DCP (5:4:1 molar ratio) at 30 mM total lipid concentration or DMPC:cholesterol:PEG2k-PE (5:4:1 molar ratio) at a total lipid concentration of either 40 mM or 60 mM.

### DETAILED DESCRIPTION

### General Considerations

For purposes of this description, certain aspects, advantages, and novel features of the embodiments of this disclosure are described herein. The disclosed methods and systems should not be construed as being limiting in any way. Instead, the present disclosure is directed toward all novel and nonobvious features and aspects of the various disclosed embodiments, alone and in various combinations and sub-combinations with one another. The methods and systems are not limited to any specific aspect or feature or combination thereof, nor do the disclosed embodiments require that any one or more specific advantages be present, or problems be solved. The technologies from any embodiment or example can be combined with the technologies described in any one or more of the other embodiments or examples. In view of the many possible embodiments to which the principles of the disclosed technology may be applied, it should be recognized that the illustrated embodiments are exemplary only and should not be taken as limiting the scope of the disclosed technology.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one skilled in the art.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person skilled in the art to have a more definitive construction, the numerical parameters set forth are approximations that may depend on the desired properties sought and/or limits of detection under standard test conditions/methods, as known to those skilled in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including 10% of that value are intended, unless explicitly stated otherwise.

Directions and other relative references may be used to facilitate discussion of the drawings and principles herein but are not intended to be limiting. For example, certain terms may be used such as “inner,” “outer,” “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part, and the object remains the same.

As used herein, “comprising” means “including,” and the singular forms “a” or “an” or “the” include plural references unless the context clearly dictates otherwise. The term “or” refers to a single element of stated alternative elements or a combination of two or more elements unless the context clearly indicates otherwise.

Although there are alternatives for various components, parameters, operating conditions, etc. set forth herein, that does not mean that those alternatives are necessarily equivalent and/or perform equally well. Nor does it mean that the alternatives are listed in a preferred order, unless stated otherwise. Unless stated otherwise, any of the groups defined below can be substituted or unsubstituted.

Unless explained otherwise, all technical and scientific terms used herein have the same meaning as commonly understood to one skilled in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Features of the presently disclosed subject matter will be apparent from the following detailed description and the appended claims.

### Overview of Terms

The following are provided to facilitate the description of various aspects of the disclosed subject matter and to guide those skilled in the art in the practice of the disclosed subject matter.

Nanoparticle: A particle formed by one or more first constituent molecules and having a maximum cross-sectional diameter (e.g., diameter) less than about 1 μm, for example, in a range of 1-200 nm, inclusive. The particle can be formed of organic or inorganic molecules and can be a vesicle or solid particle. For example, in some embodiments, the nanoparticle comprises a shell formed of lipids (i.e., a liposome). Alternatively, in some embodiments, the nanoparticle is a solid lipid particle. In some embodiments, the nanoparticle can include and/or encapsulate one or more second constituent molecules (e.g., a drug).

Drug: Any substance designed for altering one or more bodily functions of a subject when ingested, inhaled, infused, or otherwise introduced into a subject (e.g., human or animal), for example, to treat a medical condition. In some embodiments, the drug can be an aqueous-soluble substance (e.g., molecule). Alternatively or additionally, in some embodiments, the drug can be a lipid-soluble substance (e.g. molecule).

Microfluidic: Refers to flow path features in a device (e.g., inlet port, outlet port, and/or flow channel in a flow cell) and/or fluid flows within a device that have a cross-sectional dimension along a direction substantially perpendicular to or crossing a direction of the respective fluid flow less than 1 mm, for example, less than or equal to 500 μm.

Hydrocyclonic flow cell: A microfluidic flow cell having one or more axially-oriented inlet ports for providing an axial flow(s), one or more tangentially-oriented inlet ports for forming a primary vortex surrounding the axial flow(s), and one or more axially-oriented outlet ports. In some embodiments, the hydrocyclonic flow cell has a cylindrical portion or section at an inlet end, and a conical portion or section that extends from the cylindrical portion to the outlet port(s). In some embodiments, the hydrocyclonic flow cell has a tapered nozzle in the cylindrical portion that defines at least one of the axially-oriented inlet ports.

### Introduction

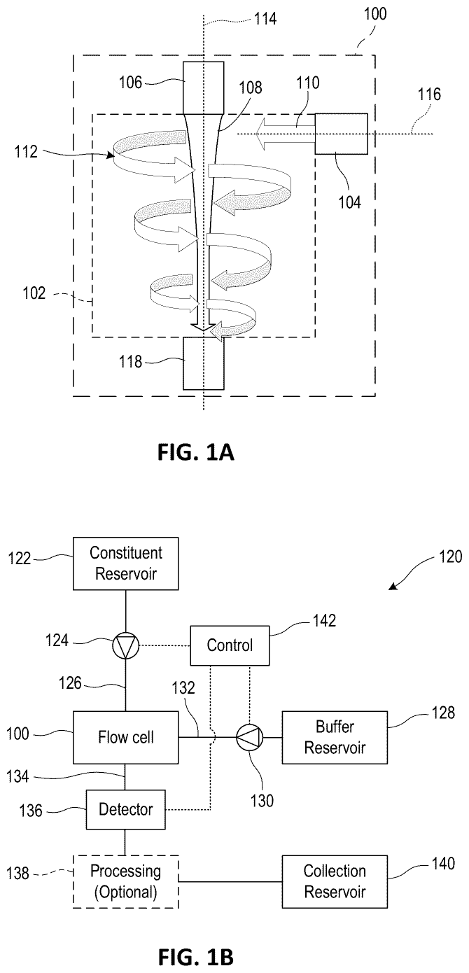

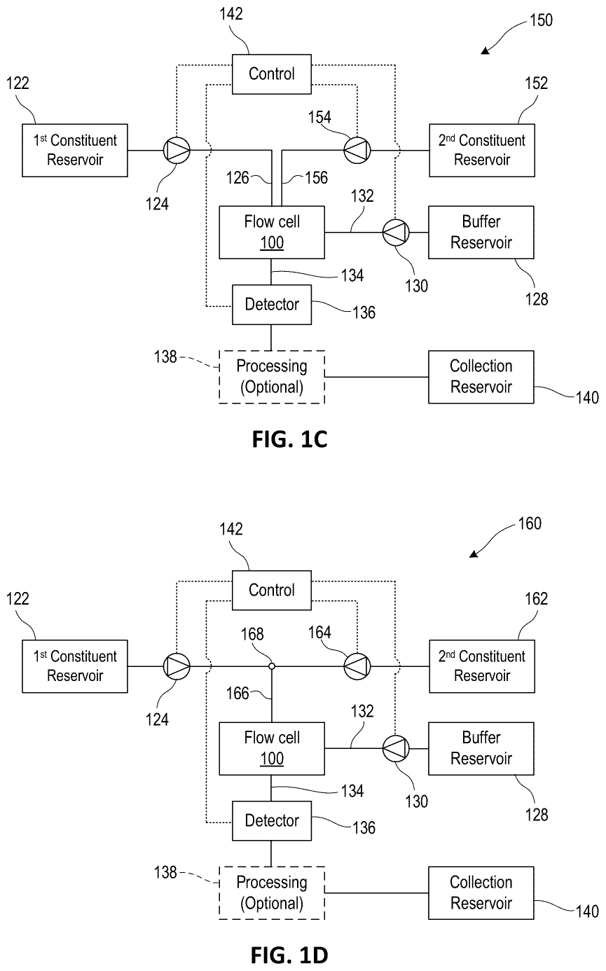

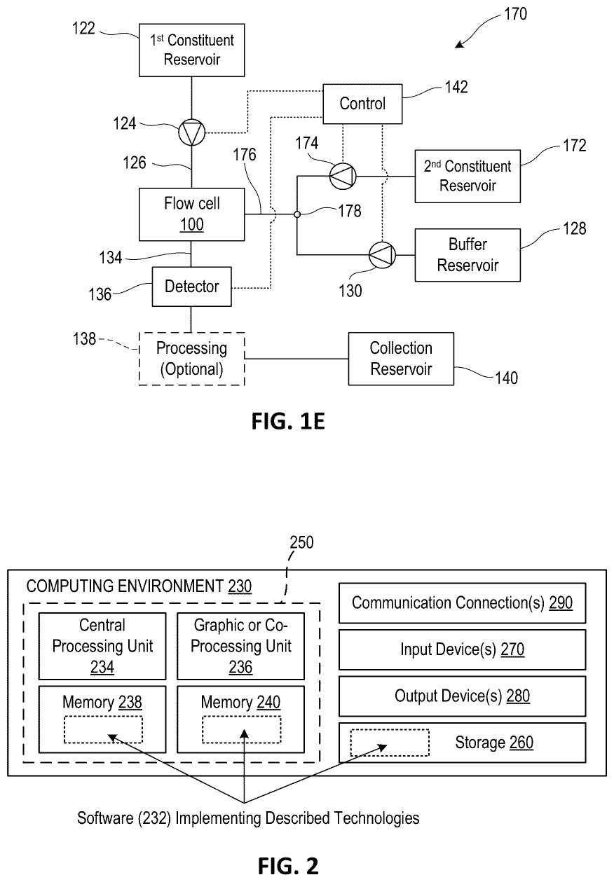

Disclosed herein are systems and methods for forming nanoparticles using microfluidic vortex focusing (MVF). In some embodiments, one or more inlet flows 110 can be directed along a respective tangential direction 116 (e.g., tangent to a circumferential direction of the mixing volume) in an internal mixing volume 102 of a hydrocyclonic flow cell 100, as shown in FIG. 1A. The one or more tangentially-directed inlet flow 110 can generate a rotational flow, or primary vortex 112, surrounding (e.g., sheathing) one or more inlet flows 108 directed along a longitudinal axis 114 (e.g., axial direction) of the mixing volume 102. The tangentially-directed inlet flow 110 and the axially-directed inlet flow 108 can be provided to the mixing volume 102 via a tangentially-oriented inlet port 104 and an axially-oriented inlet port 106, respectively.

The primary vortex 112 can subject the one or more axially-directed flows 108 to simultaneous flow focusing (e.g., in both directions in a plane perpendicular to the axial direction) and mixing within the mixing volume 102, thereby facilitating formation of nanoparticles (e.g., via self-assembly) with precise size control as the inlet flows 108, 110 proceed along the axial direction toward axially-oriented outlet port 118. In some embodiments, the sizes of the formed nanoparticles can be tuned by adjusting the respective flow rates of the tangentially-directed and axially-directed flows. At the outlet port 118, the solution flows containing the formed nanoparticles can be collected for further processing and/or use. In some embodiments, the outlet port 118 and at least one of the one or more inlet ports 106 can be aligned, for example, substantially co-axial and/or centered on longitudinal axis 114. Alternatively or additionally, the outlet port 118 can be offset from at least one of the one or more inlet ports 106, for example, along a radial direction of the mixing volume 102.

The one or more axially-directed flows 108 can have constituent molecules for forming desired nanoparticles. In some embodiments, the constituent molecules can comprise inorganic molecules or organic molecules. For example, in some embodiments, lipids can be provided in the axially-directed flow 108 (e.g., solvated lipids) to form lipid nanoparticles and/or liposomes. In some embodiments, a tangential flow 110 of buffer solution (e.g., aqueous buffer) can create the primary vortex 112 around the axial stream 108 of solvated lipids in an axisymmetric mixing volume 102. Liposome self-assembly can occur within the mixing volume 102 through the simultaneous hydrodynamic focusing and rapid convective and diffusive mixing provided by the primary vortex 112.

Vesicle formation within the mixing volume 102 is a kinetic process controlled by the local solvent polarity. As lipid solubility decreases due to a combination of solvent convection, lipid advection, and solvent/lipid co-diffusion, the amphiphilic lipid molecules can spontaneously form planar disc-like micelles. These intermediate structures can grow in a reaction rate limited process until the line energy associated with the exposed hydrophobic lipid tails overcomes the elastic energy required to form spherical vesicles, at which point membrane closure becomes energetically favorable. Because the micelle growth rate and the elastic energy at closure can depend on lipid solubility, a sharp temporal solubility gradient can enable the formation of smaller liposomes by limiting the intermediate lipid structure growth time. In the hydrocyclonic flow cell 100, stretching and folding of the fluid interface under the influence of the primary vortex 112 contributes to rapid mixing of the miscible aqueous 110 and lipid 108 streams. Meanwhile, the sheathing by the primary vortex 112 helps to spatially constrain the mixing zone, thereby significantly reducing the diffusive length scale between the aqueous 110 and lipid 108 streams.

In some embodiments, the hydrocyclonic flow cell 100 can achieve reliable liposome synthesis and narrow size distributions, for example, with vesicle diameters ranging from 61 nm to 127 nm for a lipid composition without polyethylene glycol (PEG) or as small as 27 nm when introducing a PEG-conjugated lipid in the mixture. In some embodiments, the hydrocyclonic flow cell 100 can be operated at Reynolds numbers approaching the laminar limit, for example, to yield high throughput vesicle production. For example, in some embodiments, highly monodisperse populations (e.g., having a polydispersity index (PDI)≤0.2) of liposomes with sizes less than 100 nm (e.g., as small as 27 nm in diameter) can be produced at rates of over 20 g/h. Such precise control over vesicle size during nanoscale liposome synthesis can be helpful in defining the pharmaceutical properties of liposomal nanomedicines.

Although the description above and elsewhere herein explicitly addresses synthesis of liposomes, embodiments of the disclosed subject matter are not limited thereto. Rather, the systems and methods disclosed herein can be readily adapted to synthesis of other nanoparticles (e.g., solid lipid nanoparticles, inorganic nanoparticles, etc.), according to one or more contemplated embodiments. Indeed, the techniques disclosed herein can be employed in any particle synthesis system where rapid diffusive and convective transport within a continuous-flow setup may be advantageous.

### Nanoparticle Synthesis Systems

In some embodiments, a nanoparticle synthesis system can include at least a hydrocyclonic flow cell 100 configured for MVF operation. In addition, the system can include additional modules or components to support and/or control continuous flow operation of the hydrocyclonic flow cell 100, such as, but not limited to, fluid reservoirs (e.g., supply volumes, collection volumes), fluid control components (e.g., pumps, valves, switches, conduits, etc.), detectors (e.g., particle size detectors, flow rate detectors, etc.), controllers, and/or processing systems (e.g., particle concentration systems).

For example, FIG. 1B illustrates a nanoparticle synthesis system 120 having a hydrocyclonic flow cell 100 and flow support/control components 122-142. In the illustrated example, a first reservoir 122 is fluidically coupled to an axially-oriented inlet of flow cell 100 via first inlet volume 126 (e.g., pipe, tube, or conduit), and a second reservoir 128 is fluidically coupled to an a tangentially-oriented inlet of flow cell 11 via second inlet volume 132 (e.g., pipe, tube, or conduit). In some embodiments, the first reservoir 122 can contain a solution with first constituent molecules for forming desired nanoparticles (e.g., organic and/or inorganic molecules in solution), and the second reservoir 128 can contain a buffer solution (e.g., aqueous buffer). Fluid from the first reservoir 122 can be directed by a first fluid pump 124 to the flow cell 100 via the first inlet volume 126, and the fluid from the second reservoir 128 can be directed by a second fluid pump 130 to the flow cell 100 via the second inlet volume 132.

In the illustrated example of FIG. 1B, an outlet flow with nanoparticles formed via MVF operation can be collected at an axially-oriented outlet of the hydrocyclonic flow cell 100 and directed therefrom via an outlet volume 134 (e.g., pipe, tube, or conduit). In some embodiments, the collected outlet flow may pass through an optional detection system 136, for example, to detect a size of the particles within the outlet flow. In the illustrated example of FIG. 1B, controller 142 is operatively connected to the detection system 136, the first pump 124, and the second pump 130. In some embodiments, the controller 142 can be configured to control operation of the first and second pumps 124, 130 responsively to a feedback signal from the detection system 136, for example, to tune the respective flow rates of inlet flows to the flow cell 100 (via control of pumps 124, 130) to provide a desired particle size and/or avoid undesirable constituent molecule precipitation.

In the illustrated example of FIG. 1B, the collected outlet flow from the hydrocyclonic flow cell 100 can be directed to one or more optional processing systems 138 before being directed to a collection reservoir 140. In some embodiments, the one or more processing systems 138 can prepare the nanoparticles and/or the solution containing nanoparticles for subsequent use and/or storage. For example, the one or more processing system 138 can include a concentration system, such as but not limited to an ultracentrifugation system or a centrifugal filtration system. In some embodiments, the collection reservoir 140 can hold the solution with nanoparticles therein for subsequent use (e.g., without further processing), for further processing (e.g., concentration, particle modification, etc.), or storage (e.g., at a reduced temperature, such as about ˜4° C.).

In the discussion of FIG. 1B above, the first reservoir is described as containing a solution with the first constituent molecules for forming desired nanoparticles. However, in some embodiments, the solution in the first reservoir may also contain second constituent molecules, and the resulting nanoparticles can comprise both the first and second constituent molecules, for example, with the first constituent molecules forming a shell encapsulating the second constituent molecules (e.g., a vesicle, such as a liposome) or with the second constituent molecules being encapsulated by or embedded within the first constituent molecules (e.g., a solid lipid nanoparticle). In such embodiments, the second constituent molecules may be soluble in the first constituent molecules and/or the solution in the first reservoir. For example, the first constituent molecules can comprise lipids, and the second constituent molecules can comprise a lipid-soluble drug.

Alternatively, in some embodiments, the second constituent molecules can be provided with a separate reservoir and/or inlet flow volume into the flow cell 100. For example, FIG. 1D illustrates a nanoparticle synthesis system 160 with a first reservoir 122 containing a solution with first constituent molecules and a third reservoir 162 containing a solution with second constituent molecules. In the illustrated example of FIG. 1D, fluid from the first reservoir 122 can be directed by first fluid pump 124 to a junction 168 (e.g., union, switch, etc.), and fluid from the third reservoir 162 can be simultaneously or sequentially directed by a third fluid pump 164 to the junction 168. The first and second constituent molecules can thus be combined at junction 168 before being introduced to an axially-oriented inlet of flow cell 100 via inlet volume 166 (e.g., pipe, tube, or conduit). In such embodiments, the second constituent molecules may be soluble in the first constituent molecules and/or the solution from the first reservoir. For example, the first constituent molecules can comprise lipids, and the second constituent molecules can comprise a lipid-soluble drug.

In the example of FIG. 1D, the second constituent molecules are introduced to the flow cell 100 via the same axially-oriented inlet as the first constituent molecules. Alternatively, in some embodiments, the second constituent molecules can be introduced via one or more axially-oriented inlets separate from that for the first constituent molecules. For example, FIG. 1C illustrates a nanoparticle synthesis system 150 with a first reservoir 122 containing a solution with first constituent molecules and a third reservoir 152 containing a solution with second constituent molecules. Similar to the example of FIG. 1B, fluid from the first reservoir 122 can be directed by first fluid pump 124 along first inlet volume 126 to a first axially-oriented inlet (e.g., substantially coaxial with a central axis of the mixing volume) of flow cell 100. Simultaneously or sequentially, fluid from the third reservoir 152 can be directed by third fluid pump 154 along a separate inlet volume 156 (e.g., pipe, tube, or conduit) to another axially-oriented inlet (e.g., an annular port substantially coaxial with a central axis of the mixing volume, or one or more ports parallel to but offset from the central axis) of flow cell 100. The flows of first and second constituent molecules can thus be initially adjacent within the mixing volume of flow cell 100 and surrounded by the primary vortex. In such embodiments, the second constituent molecules may be soluble in the buffer solution from the second reservoir 128, but not necessarily soluble in the first constituent molecules and/or the solution from the first reservoir. For example, the first constituent molecules can comprise lipids, and the second constituent molecules can comprise an aqueous-soluble drug.

In the discussion of FIG. 1B above, the second reservoir is described as containing buffer solution. However, in some embodiments, the buffer solution in the second reservoir may also contain second constituent molecules, and the resulting nanoparticles can comprise both the first and second constituent molecules, for example, with the first constituent molecules forming a shell encapsulating the second constituent molecules (e.g., a vesicle, such as a liposome) or with the second constituent molecules being encapsulated by or embedded within the first constituent molecules (e.g., a solid lipid nanoparticle). In such embodiments, the second constituent molecules may be soluble in the buffer solution in the second reservoir. For example, the first constituent molecules can comprise lipids, and the second constituent molecules can comprise an aqueous-soluble drug.

Alternatively, in some embodiments, the second constituent molecules can be provided with a separate reservoir and/or inlet flow volume into the flow cell 100. For example, FIG. 1E illustrates a nanoparticle synthesis system 170 with a second reservoir 128 containing buffer solution and a third reservoir 172 containing a solution with second constituent molecules. In the illustrated example of FIG. 1E, fluid from the second reservoir 128 can be directed by second fluid pump 130 to a junction 178 (e.g., union, switch, etc.), and fluid from the third reservoir 172 can be simultaneously or sequentially directed by a third fluid pump 174 to the junction 178. The second constituent molecules and buffer can thus be combined at junction 178 before being introduced to a tangentially-oriented inlet of flow cell 100 via inlet volume 176 (e.g., pipe, tube, or conduit). Alternatively, in some embodiments, the second constituent molecules can be introduced via one or more tangentially-oriented inlets separate from that of the buffer solution. In such embodiments, the second constituent molecules may be soluble in the buffer solution in the second reservoir. For example, the first constituent molecules can comprise lipids, and the second constituent molecules can comprise an aqueous-soluble drug.

In some embodiments, for example, in any of the illustrated examples of FIGS. 1B-1E, one, some, or all of the reservoirs can be replaced with a respective dynamic flow volume, for example, a conduit for a continuous flow from an upstream first constituent molecule (e.g., lipid) production system, an upstream second constituent molecule (e.g., drug) production system, or an upstream buffer production system. In some embodiments, for example, in any of the illustrated examples of FIGS. 1B-1E, one, some, or all of the fluid pumps can be combined with its respective source reservoir, for example, when implemented as a syringe pump or similar configuration. In some embodiments, for example, in any of the illustrated examples of FIGS. 1B-1E, one, some, or all of the fluid pumps may instead be arranged upstream from the respective source reservoir. In some embodiments, for example, in any of the illustrated examples of FIGS. 1B-1E, one, some, or all of the fluid pumps can be replaced by a non-mechanical flow-inducing mechanism (such as but not limited to applying pressure upstream of the respective reservoir) and/or can be supplemented by other flow control components (e.g., valves, switches, etc.). In some embodiments, for example, in any of the illustrated examples of FIGS. 1B-1E, one, some, or all of the fluid pumps may be omitted, for example, when flow is provided directly from an upstream production system.

### Computer Implementation

FIG. 2 depicts a generalized example of a suitable computing environment 230 in which the described innovations may be implemented, such as but not limited to aspects of controller 142 and/or a nanoparticle synthesis method. The computing environment 230 is not intended to suggest any limitation as to scope of use or functionality, as the innovations may be implemented in diverse general-purpose or special-purpose computing systems. For example, the computing environment 230 can be any of a variety of computing devices (e.g., desktop computer, laptop computer, server computer, tablet computer, etc.).

With reference to FIG. 2, the computing environment 230 includes one or more processing units 234, 236 and memory 238, 240. In FIG. 2, this basic configuration 250 is included within a dashed line. The processing units 234, 236 execute computer-executable instructions. A processing unit can be a central processing unit (CPU), processor in an application-specific integrated circuit (ASIC), or any other type of processor (e.g., hardware processors, graphics processing units (GPUs), virtual processors, etc.). In a multi-processing system, multiple processing units execute computer-executable instructions to increase processing power. For example, FIG. 2 shows a central processing unit 234 as well as a graphics processing unit or co-processing unit 236. The tangible memory 238, 240 may be volatile memory (e.g., registers, cache, RAM), non-volatile memory (e.g., ROM, EEPROM, flash memory, etc.), or some combination of the two, accessible by the processing unit(s). The memory 238, 240 stores software 232 implementing one or more innovations described herein, in the form of computer-executable instructions suitable for execution by the processing unit(s).

A computing system may have additional features. For example, the computing environment 230 includes storage 260, one or more input devices 270, one or more output devices 280, and one or more communication connections 290. An interconnection mechanism (not shown) such as a bus, controller, or network interconnects the components of the computing environment 230. Typically, operating system software (not shown) provides an operating environment for other software executing in the computing environment 230, and coordinates activities of the components of the computing environment 230.

The tangible storage 260 may be removable or non-removable, and includes magnetic disks, magnetic tapes or cassettes, CD-ROMs, DVDs, or any other medium which can be used to store information in a non-transitory way, and which can be accessed within the computing environment 230. The storage 260 can store instructions for the software 232 implementing one or more innovations described herein.

The input device(s) 270 may be a touch input device such as a keyboard, mouse, pen, or trackball, a voice input device, a scanning device, or another device that provides input to the computing environment 230. The output device(s) 280 may be a display, printer, speaker, CD-writer, or another device that provides output from computing environment 230.

The communication connection(s) 290 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, radio-frequency (RF), or another carrier.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or any other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java™, Python®, and/or any other suitable computer language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. In any of the above-described examples and embodiments, provision of a request (e.g., data request), indication (e.g., data signal), instruction (e.g., control signal), or any other communication between systems, components, devices, etc. can be by generation and transmission of an appropriate electrical signal by wired or wireless connections.

### Hydrocyclonic Flow Cells

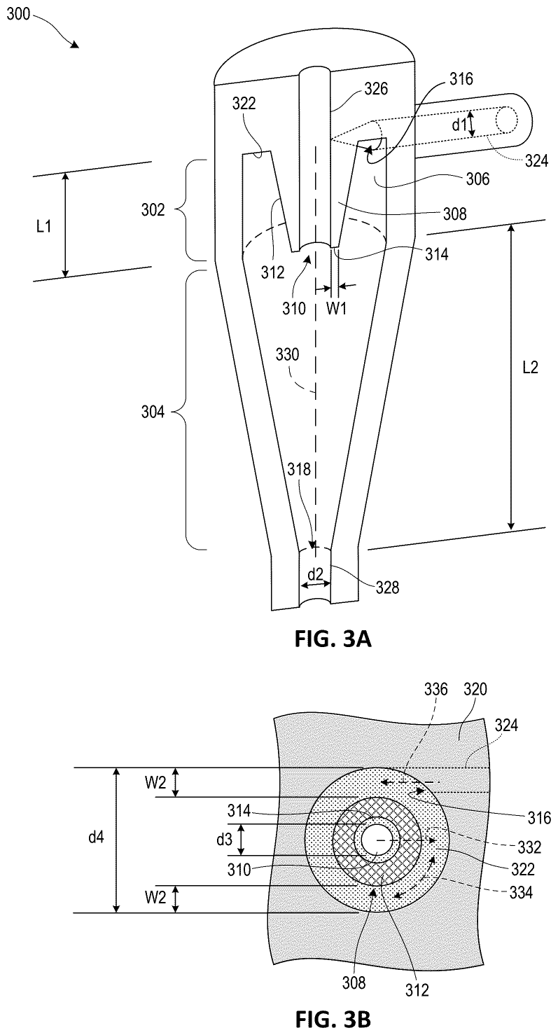

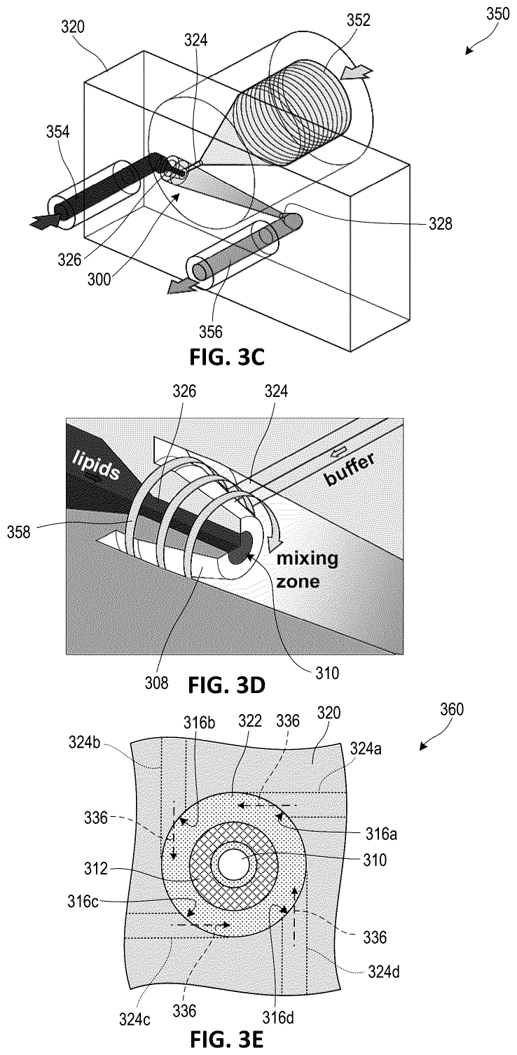

Referring to FIGS. 3A-3B, an exemplary configuration of a hydrocyclonic flow cell 300 is shown. The hydrocyclonic flow cell 300 can have a cylindrical portion or section 302 and a conical portion or section 304. For example, the cylindrical and conical portions 302, 304, and/or fluid conduits connecting thereto (e.g., inlet conduit 324, inlet conduit 326, and outlet conduit 328) can be defined within a bulk material 320 (e.g., as shown in FIGS. 3B-3C). In some embodiments, the bulk material 320 can be a material compatible with the molecules and solvents flowed therein for particle synthesis, for example, a polymer (e.g., acrylic-based photosensitive resin such as HTM140).

The hydrocyclonic flow cell 300 can further include an inlet nozzle 308 that defines an axially-oriented inlet port 310, for example, for introducing a flow of first constituent molecules (e.g., lipids in solution) along the longitudinal axis 330 of the flow cell 300 toward axially-oriented outlet port 318. In the illustrated example, the inlet port 310 and the outlet port 318 are coaxial and aligned with the longitudinal axis 330 of the flow cell 300. In some embodiments, the cylindrical portion 302 can have a circumferential wall 306 extending substantially parallel to the longitudinal axis 330 from a base wall 322 and surrounding the inlet nozzle 308 in plan view, thereby defining an annular inlet region to which a tangentially-oriented inlet port 316 opens. Fluid (e.g., provided via conduit 324) can exit inlet port 316 along a tangent 336 and flow through the annular inlet region of the cylindrical portion 302 along circumferential direction 334, thereby providing a primary vortex surrounding a flow (e.g., provided via conduit 326) exiting from inlet port 310.

In some embodiments, the nozzle 308 can have a tapered radially-outer surface 312 (e.g., along the axial direction), for example, to reduce a distance between the annular vortex generation zone and the axial flow from inlet port 310 for enhanced mixing. In the illustrated example of FIGS. 3A-3B, the nozzle 308 is tapered to a substantially flat annular tip 314 defining the inlet port 310. For example, the inlet port 310 can have a diameter, d3, less than or equal to about 0.5 mm (e.g., about 300 μm or less), and the tip 314 can have a width, W1, along radial direction 332 less than or equal to about 50% of d3 (e.g., about 150 μm or less). However, in some embodiments, W1 can be much less d3, for example, approaching a point or thin line (e.g., W1≤10 μm). Alternatively or additionally, in some embodiments, the nozzle may not be tapered along its length, for example, where the radially-outer surface 312 extends substantially parallel to the axial direction. In some embodiments, the inlet port 316 can have a diameter, d1, that is the same or different than d3, for example, less than or equal to about 0.5 mm (e.g., about 300 μm or less). Alternatively or additionally, in some embodiments, the outlet port 318 can have a diameter, d2, that is the same or different than d3, for example, less than or equal to about 0.5 mm (e.g., about 300 μm or less).

In some embodiments, the cylindrical portion 302 can have a length, L1, along the axial direction (e.g., parallel to longitudinal axis 330), and the nozzle 308 can extend across the entire length, L1, or terminate before the conical portion 304. In some embodiments, L1 of the cylindrical portion 302 can be less than a length, L2, of the conical portion 304 along the axial direction. For example, L1 (e.g., about 1 mm) may be less than or equal to 20% of L2 (e.g., about 7.4 mm). In some embodiments, the annular inlet region formed between the wall 306 and the junction between the nozzle 308 and base wall 322 can have a width, W2, along the radial direction 332. In some embodiments, the width, W2, can be the same or different than d3, for example, less than or equal to about 0.5 mm (e.g., about 300 μm or less).

In some embodiments, the hydrocyclonic flow cell 300 can be integrated in a nanoparticle synthesis system 350, such as illustrated in FIGS. 3C-3D. In some embodiments, the same bulk material 320 used to form the features of the hydrocyclonic flow cell 300 can be used to form fluidic network features of the nanoparticle synthesis system 350, for example, a first solution reservoir 352 (e.g., for buffer solution), a second solution reservoir 354 (e.g., for lipid solution), and/or nanoparticle collection reservoir 356. The first solution reservoir 352 can be fluidically coupled to the inlet conduit 324 to provide a flow of buffer solution to tangentially-oriented inlet port 316, and the second solution reservoir 354 can be fluidically coupled to the inlet conduit 326 to provide a flow of solution containing the first constituent molecules (e.g., lipids) to the axially-oriented inlet port 310. The nanoparticle collection reservoir 356 can be fluidically coupled to the outlet conduit 328 to receive a flow of solution with synthesized nanoparticles therein.

In some embodiments, system 350 with hydrocyclonic flow cell 300 can be used to synthesize liposomes. As shown in FIG. 3D, a vortical flow field during focusing can be generated by tangentially injecting aqueous buffer into the upper annular region of an axisymmetric chamber, resulting in a spiral flow path 358 that continues into the lower conical mixing chamber. Meanwhile, lipid solution can be injected through the axially-oriented inlet port 310 within the vortex generation zone. The lipid solution can emerge into the center of the mixing chamber sheathed by the rotating buffer flow, which can transfer rotational momentum to the lipid solution. To promote efficient focusing, the inner surface of the annular structure (e.g., surface 312 of the nozzle 308) can be tapered to minimize geometric discontinuity at the fluid junction. After mixing within the conical chamber, the combined flow with nanoparticles therein can exit through lower fluid port 318 for collection.

The fluid mixing can occur when multiple fluid elements are brought into contact, allowing scalar concentrations within each volume to diffuse across the interface. In the case of a 2D steady vortex flow, local shear results in fluid elements located on different streamlines to separate, thereby stretching the fluid interface and folding adjacent elements around one another. For 3D vortex focusing generated by the hydrocyclonic flow cell 300 of FIGS. 3A-3D, hydrodynamic focusing of the lipids during injection into the vortex flow serves to shrink the initial axial cross section of the lipid stream, thus minimizing the radial length scale of the stretched fluid volumes. This process serves to reduce the diffusion length scale and mixing times for the polar and aqueous solvents as well as the lipid solutes themselves.

In the illustrated examples of FIGS. 3A-3D, a single tangentially-oriented inlet 316 for injecting a buffer solution into the cylindrical portion 302 is shown. Alternatively, in some embodiments, the cylindrical portion 302 can be provided with two or more tangentially-oriented inlets, for example, for simultaneous or sequential injection of solution at different locations around the circumference of wall 306. For example, FIG. 3E illustrates an exemplary configuration 360 for the cylindrical portion of a hydrocyclonic flow cell with four tangentially-oriented inlet ports 316a-316d. In some embodiments, the inlet ports 316a-316d can be equidistantly spaced around the circumference of the cylindrical portion. In the illustrated example, each inlet port 316a-316d may be fluidically coupled to a respective inlet conduit 324a-324d for providing buffer solution (or another solution, for example, containing second constituent molecules, such as an aqueous-soluble drug) thereto. Alternatively or additionally, one, some, or all of the inlet ports 316a-316d can be fluidically coupled together, for example, via one or more junctions upstream of inlet conduits 324a-324d.

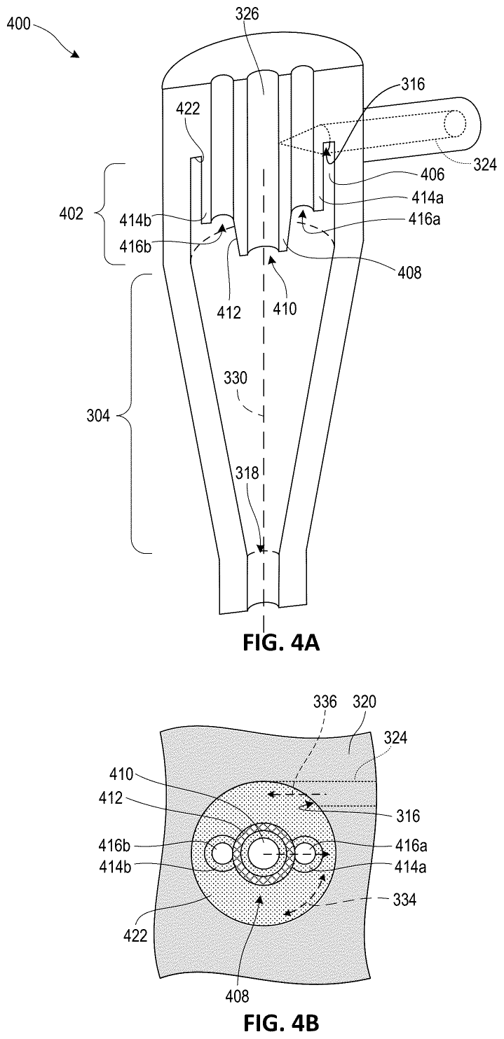

In the illustrated examples of FIGS. 3A-3E, a single axially-oriented inlet 310 for injecting a solution of first constituent molecules is shown. Alternatively, in some embodiments, the hydrocyclonic flow cell can be provided one or more additional axially-oriented inlets, for example, for simultaneous or sequential injection of solution (e.g., containing second constituent molecules) at locations adjacent to the axial flow of first constituent molecules. For example, FIGS. 4A-4B illustrate an exemplary hydrocyclonic flow cell 400 with additional axially-oriented inlet ports, in particular, a pair of secondary inlet ports 416a, 416b on opposite sides of axially-oriented inlet port 410. Similar to the flow cell 300 of FIGS. 3A-3E, the axially-oriented inlet port 410 can be formed at the end of inlet nozzle 408 having a tapered, radially-outer surface 412, and the inlet nozzle 408 can be surrounded by circumferential wall 406 extending along the axial direction from base wall 422 of the cylindrical portion 402.

Each secondary inlet port 416a, 416b can be formed via respective internal axially-extending walls 414a, 414b, for example, extending substantially parallel to the longitudinal axis 330. Alternatively, in some embodiments, walls 414a, 414b can be tapered, for example in a manner similar to that of nozzle 408. In the illustrated example of FIG. 4A, the lengths of walls 414a, 414b along the axial direction is less than that of the nozzle 408 and the cylindrical portion 402. Alternatively, in some embodiments, the length of walls 414a, 414b and/or the nozzle 408 can be such that inlet port 410 and the secondary inlet ports 416a, 416b are at substantially the same axial location or such that the secondary inlet ports 416a, 416b are downstream from the inlet port 410. Although only two secondary inlet ports are shown in FIGS. 4A-4B, any number of secondary inlet ports disposed around nozzle 408 is possible according to one or more contemplated embodiments. The one or more secondary inlet ports can be used to introduce a respective flow of second constituent molecules (e.g., drug to be encapsulated) adjacent to the flow of first constituent molecules (e.g., lipids in solution) from inlet port 410 along the longitudinal axis 330.

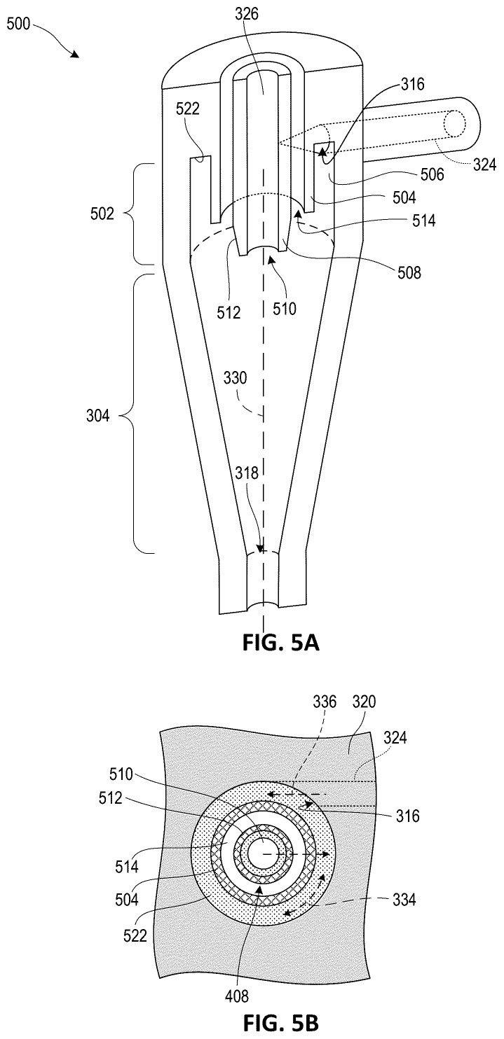

Alternatively, in some embodiments, the provision of secondary inlet ports can be extended to cover an entire circumference around the inlet nozzle, for example, by providing an annular port. For example, FIGS. 5A-5B illustrate an exemplary hydrocyclonic flow cell 500 with an additional axially-oriented inlet port, in particular, an annular secondary inlet port 514 centered on and surrounding axially-oriented inlet port 510. Similar to the flow cell 300 of FIGS. 3A-3E, the axially-oriented inlet port 510 can be formed at the end of inlet nozzle 508 having a tapered, radially-outer surface 512, and the inlet nozzle 508 can be surrounded by circumferential wall 506 extending along the axial direction from base wall 522 of the cylindrical portion 502.

The secondary inlet port 514 can be formed via internal axially-extending wall 504, for example, extending substantially parallel to the longitudinal axis 330. Alternatively, in some embodiments, wall 504 can be tapered, for example, in a manner similar to that of nozzle 508. In the illustrated example of FIG. 5A, the length of wall 504 along the axial direction is less than that of the nozzle 508 and the cylindrical portion 502. Alternatively, in some embodiments, the length of wall 504 and/or the nozzle 508 can be such that inlet port 510 and the secondary inlet port 514 are at substantially the same axial location or such that the secondary inlet port 514 is downstream from the inlet port 510. Although only a single annular secondary inlet port is shown in FIGS. 5A-5B, any number of annular secondary inlet ports centered on nozzle 508 is possible according to one or more contemplated embodiments. The secondary inlet port can be used to introduce a respective annular flow of second constituent molecules (e.g., drug to be encapsulated) adjacent to and surrounding the flow of first constituent molecules (e.g., lipids in solution) from inlet port 510 along the longitudinal axis 330.

### Fabricated Examples and Experimental Results

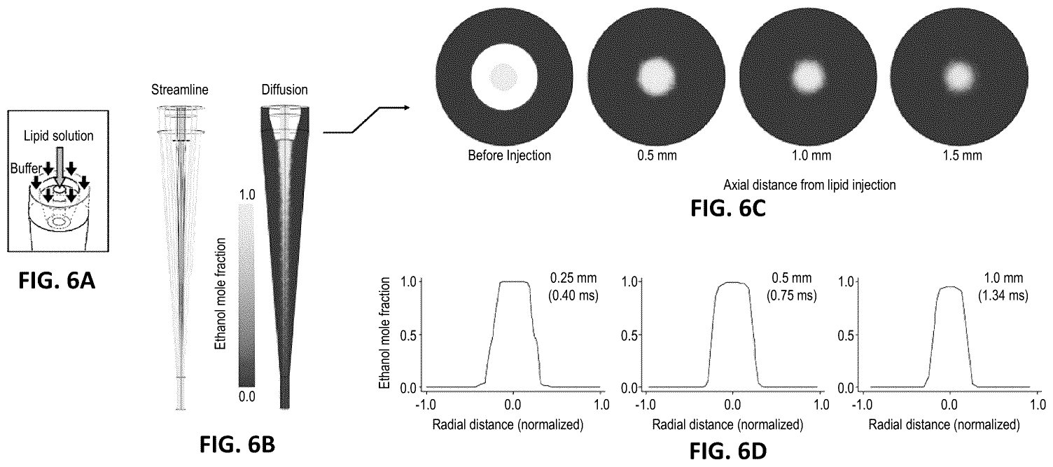

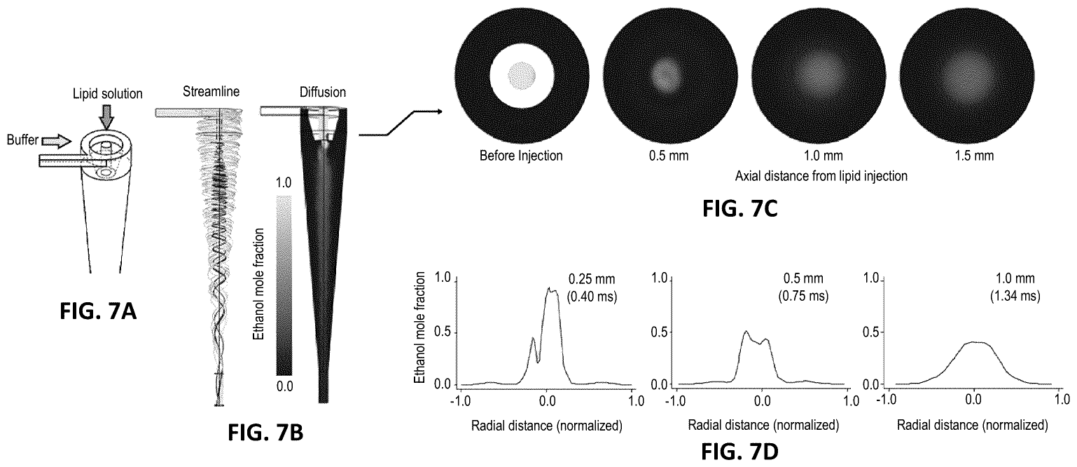

To study the combined mixing and focusing offered by the disclosed MVF technique, a numerical model was developed to investigate the distribution of solvent during both hydrodynamic flow focusing (HFF) and MVF, with results summarized in FIGS. 6A-6D and 7A-7D, respectively. The HFF (FIG. 6A) was performed using the same device geometry as MVF (FIG. 7A), but with the aqueous buffer injected axially (instead of tangentially) into the annular region, thereby avoiding vortex formation. For both device configurations, the evolution of the solvent concentration profile was a primary factor in predicting the final liposome size. Vesicle formation began when reduced lipid solubility leads to the formation of small bilayer fragments that continue to grow in a rate-limited process until vesicle enclosure becomes energetically favorable.

Light scattering was employed to evaluate particle anisotropy as the solvent concentration was reduced. A sharp decrease in dissymmetry and increase in depolarization was observed at a solvent mole fraction of 0.5, consistent with the formation of disk-like structures around this value of ethanol concentration. Based on this observation, a mole fraction of 0.5 was adopted as an appropriate threshold below which vesicle formation begins to occur in the experiments. As shown in FIGS. 6B-6D for the case of HFF, the peak ethanol concentration remains at a value near unity at a point 1.5 mm downstream of the fluid junction, and only reaches the selected threshold value of 0.5 after travelling an axial distance of 3.1 mm. In contrast, as shown in FIGS. 7B-7D for the case of MVF, the peak ethanol mole fraction drops to approximately 0.5 within 500 μm of the focusing junction. For the bulk buffer and lipid flow rates used in this study, the threshold concentration during MVF was reached within 0.75 ms after entering the mixing zone, compared with 2.50 ms for the case of HFF. The significantly faster mixing associated with the MVF process limits the time available for lipid fragment growth, and thus can enable the synthesis of smaller vesicles.

Nanoparticle synthesis systems with miniature hydrocyclonic flow cell devices were fabricated and used for nanoscale liposome synthesis via the disclosed MVF technique. Devices were fabricated using a high-resolution stereolithography digital light processing (SLA-DLP) process. Each device had a total chamber diameter (e.g., d4 in FIGS. 3A-3B) of 1.5 mm, a length (e.g., L1+L2 in FIGS. 3A-3B) of 8.4 mm, a vortex formation gap (e.g., W2 in FIGS. 3A-3B) of 300 μm, and inlet/outlet channel diameters (e.g., d1, d2, and d3 in FIGS. 3A-3B) of 300 μm. To minimize the fluid dead volume during lipid focusing, the lipid injection channel was tapered to a final tip thickness (e.g., W1 in FIGS. 3A-3B) of 150 μm. A threaded port design was employed for the buffer inlet to support the use of an Upchurch F-120 fitting (sold by IDEX Health & Science, Oak Harbor, WA) for high-pressure fluidic connection.

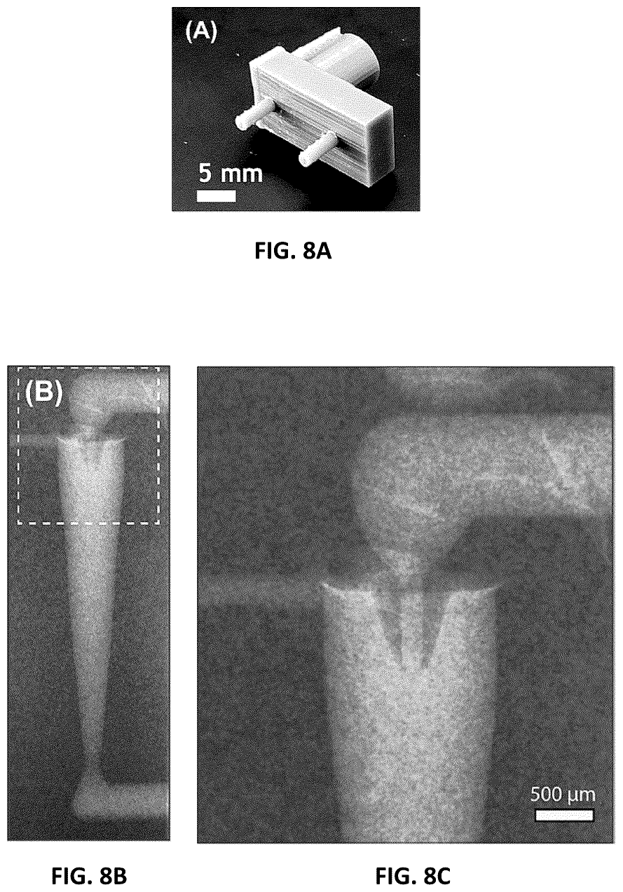

The resulting stereolithography (STL) file was converted into a mask layer stack for SLA-DLP printing with 25 μm z-step on a Perfactory 4 DLP-SLA instrument (sold by EnvisionTEC Inc., Dearborn, MI) using the EnvisionTEC Magics software. The stereolithography tool was equipped with a 75 mm objective lens for high-resolution printing, corresponding to a 74 mm×46 mm printable area at 1920×1200 pixel resolution. The STL file was oriented to align the axial center of the hydrocyclonic flow cell perpendicular to the print stage. After printing, the system was developed, processed (e.g., cleaned to remove residue) and cured (e.g., using an Otoflash UV curing unit sold by EnvisionTEC Inc., Dearborn, MI). FIGS. 8A-8C shows an example of a fabricated nanoparticle synthesis system with hydrocyclonic flow cell having the configuration of FIGS. 3A-3D.

Print orientation was found to play a role in realizing the tapered annular structure that serves as a nozzle for the lipid inflow. The axial direction of the hydrocyclonic flow cell was thus aligned perpendicular to the print stage, with the upper ceiling of flow cell facing the print stage, allowing the thicker base of the tapered annular structure to be formed before patterning the tapering geometry. In addition, since mixing occurs at the junction of the annular vortex generation zone and the annular lipid inlet, nozzle asymmetry may affect the flow and mixing profiles during focusing. While orienting the devices perpendicular to the stage during printing helped to reduce asymmetry, nozzle deformation was further minimized by defining a fixed thickness for the nozzle tip, rather than allowing the geometry to taper to a point. A tip thickness (e.g., W1 in FIG. 3A) of 150 μm was found to be the minimum dimension that could be reliably formed for the particular printing tool and resin used in this work; however, thinner tips should be possible via different fabrication systems and/or techniques.

During process optimization, the lipid injection channel diameter and wall thickness were minimized with the goal of reducing the radial mixing length scale during liposome formation. While channel dimensions as small as 150 μm were investigated, a diameter of 300 μm was selected for the final flow cells, since smaller ports were routinely found to be closed prior to the final development step. Designing the nozzle to have a length less than 1 mm also helped to avoid warping or clogging. However, smaller channels and/or longer nozzles should be possible via different fabrication systems and/or techniques. Finally, the light intensity during stereolithography was carefully controlled to improve device geometry and performance, in particular, by limiting exposure intensity to 97% of the nominal instrument level. Under the optimized processing conditions, device yield was approximately 50%, with full or partial clogging of the lipid channel being the primary failure mode. Surface roughness within the hydrocyclonic flow cell was measured by optical profilometry after cutting open the chamber using a low speed saw, with average roughness values of Ra=1.32 μm and Ra=0.45 μm observed in the axial and radial directions, respectively.

Characteristics of the resin used for device fabrication can also impact device performance. To support liposome synthesis, the material should be compatible with the solvent used as a lipid carrier and offer sufficient rigidity and mechanical strength to avoid deforming or breaking during high pressure operation. To ensure compatibility with ethanol as the lipid solvent, an acrylic-based photosensitive resin (HTM140, sold by EnvisionTEC of Dearborn, Michigan)) was selected. Secondary UV exposure to fully polymerize the resin after development can be used to prevent solvent-induced mechanical failure, such as cracking due to the presence of unreacted monomer, oligomers, or low molecular weight polymers within the solidified resin.

Particle diameter and size distribution can be important for controlling therapeutic effect and safety for nanocarrier systems. To emphasize the importance of controlling vesicle size and polydispersity for liposomal nanomedicines, consider a liposome population with known mean diameter and polydispersity index (PDI), defined as the particle size variance normalized by the square of the mean diameter. The volume of hydrophilic drug encapsulated within the liposome core for a given vesicle size range can be determined by integrating the product of the distribution probability density function and size-dependent particle volume. Given a log-normal particle size distribution with location and scale parameters derived from mean diameter and PDI values, a population with a mean diameter of 100 nm and PDI of 0.2 was found to have approximately 80% of the total drug encapsulated by the liposomes retained within particles larger than 100 nm, i.e., within a size range where delivery to the targeted tissues may not be optimal and accumulation in healthy organs can occur. In contrast, reducing the size to 80 nm and PDI to 0.05 significantly reduces the drug associated with these larger particles to below 28%. A similar analysis applies for the case of hydrophobic drug intercalated within the liposome membrane, where drug amount scales approximately with membrane area. In this case, 67% of the drug is found to be retained in vesicles above 100 nm for the larger and more polydisperse vesicle population, compared with only 21% for the smaller and more uniform particles.

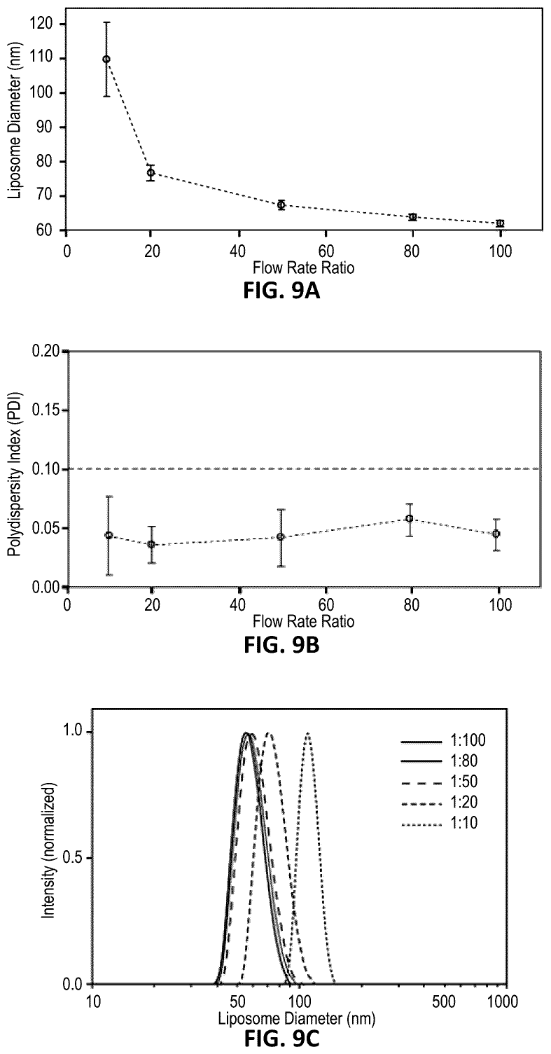

The size of liposomes generated by MVF can be directly controlled by adjusting the relative flowrates of solvated lipid and aqueous buffer injected into the system. The impact of the buffer:lipid flow rate ratio (FRR) on liposome size and size distribution is shown in FIGS. 9A-9C. Using a constant total flow rate of 60 mL/min and 10 mM lipid concentration in the injected ethanol stream, increasing the flow rate ratio over a full log from 10 to 100 led to a significant decrease in liposome size, with a minimum diameter of 61 nm at FRR=100 (FIG. 9A). The process was highly repeatable, with minimal variation in mean diameter observed over 6 replicates for each liposome synthesis condition.

The inverse relationship between FRR and vesicle size was similar to liposome formation using HFF or rapid micromixing; however, the MVF technique was found to yield lower polydispersity, with an average PDI value of 0.04 and nearly constant size variance over the full range of flow rate ratios (FIG. 9B). A production figure of merit (Q) can be defined as the inverse of the product of liposome diameter (d) and polydispersity (PDI):

Q=d⁻¹PDI⁻¹.

For the MVF platform, the Q values were generally in the range of 0.1-0.4. In contrast, conventional techniques such as ethanol injection or chaotic advection micromixing yielded Q values below 0.15.

A potential limitation of the MVF technique is the use of high FRR values to reduce vesicle size. High FRR may necessitate subsequent concentration of the liposome solutions for clinical use and/or may impact drug encapsulation efficiency. In practice, liposome synthesis techniques may employ downstream processing for buffer exchange, filtration, and concentration adjustment. To concentrate dilute suspensions of larger liposomes, ultracentrifugation can be employed. Smaller nanoliposomes may be concentrated by centrifugal filtration using a filter element with an appropriate cutoff size, which may also serve to remove solvent and free drug from the final liposome suspension.

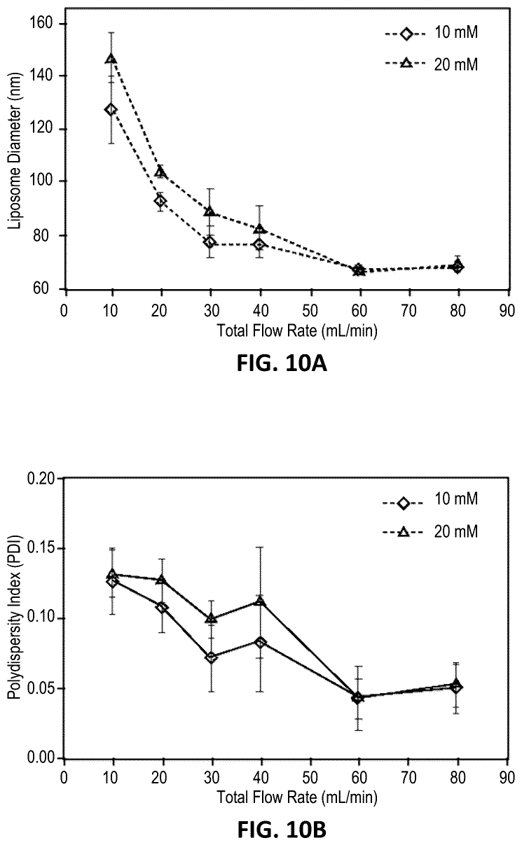

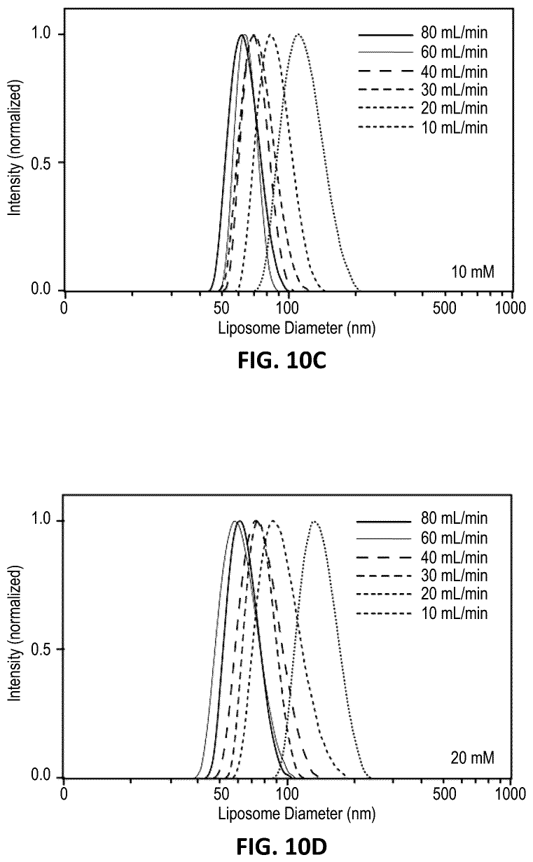

When operating the MVF system at constant FRR, the overall liposome production throughput may be enhanced by increasing the total flow rate (TFR) of the combined lipid and buffer flows through the system. The impact of TFR on liposome size distribution is shown in FIGS. 10A-10D. With the flow ratio held at a constant FRR value of 50, a strong inverse relationship between total flow rate and both liposome size and PDI was observed (FIGS. 10A-10B). This differs from the reported behavior of other microfluidic techniques such as HFF and chaotic advection mixing, where vesicle size and polydispersity were insensitive to TFR. This difference may be explained by the influence of vortical flow on mass transport within the hydrocyclonic flow cell. At higher aqueous buffer flow rates, the rotational flow velocity surrounding the injected lipid solution increases, thereby enhancing mixing during the focusing process. The same behavior is not observed in either HFF or chaotic advection since the mixing streamlines in these techniques are invariant with total flow rate.

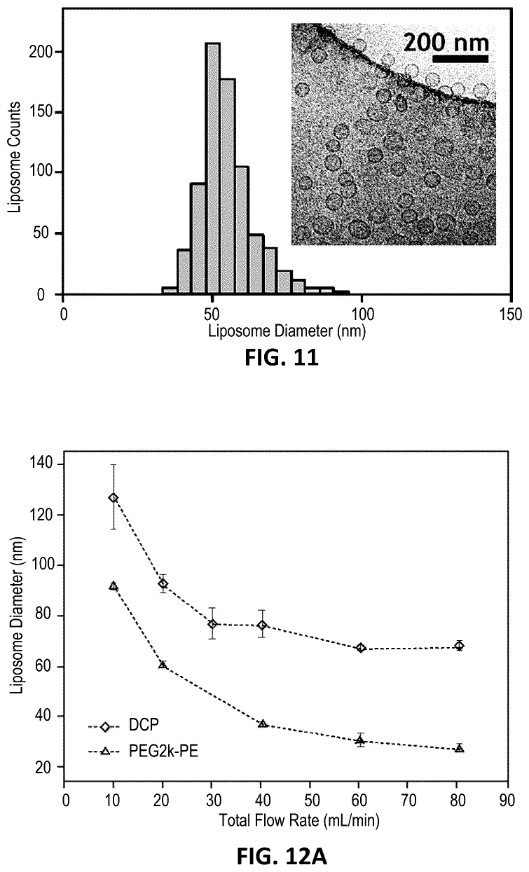

The impact of lipid concentration on liposome formation is also shown in FIGS. 10A-10D. A small increase in both mean diameter and PDI was observed when raising the concentration of lipids in the feed stream from 10 mM to 20 mM. Measured size distributions (measured via dynamic light scattering (DLS)) for the particles generated under both lipid concentrations are provided in FIGS. 10C-10D. A representative size distribution generated by direct characterization of a liposome sample imaged by Cryo-TEM is shown in FIG. 11 for comparison with the DLS data of FIGS. 10C-10D.

Long-term storage of liposomes is also a consideration for nanomedicine applications. Structural instability driven by thermodynamic perturbations can lead to degradation and structural reorganization of the vesicles. To evaluate the colloidal stability of vesicles generated by the MVF technique, three selected liposome populations formed under different total flow rates and flow rate ratios were stored at 4° C. for 99 days, with size distributions measured before and after storage. During this time period no detectable change in mean particle size or size variance was observed for any of the samples

Liposome surface modifications can also impact pharmacokinetic properties. For example, the attachment of polyethylene glycol (PEG) to the outer liposome surface allows the nanoparticles to avoid recognition by the MPS, thereby enabling longer blood circulation times, improved bioavailability, and higher levels of accumulation in tumor tissues. Because the presence of large PEG molecules imposes steric effects during liposome formation, it was desirable to understand the relationship between PEG content and vesicle size. The inclusion of PEG-conjugated lipids during nanoparticle formation can stabilize the resulting particles. While an inverse relationship between particle size and both PEG concentration and PEG chain length has been reported for the case of solid lipid nanoparticle nucleation, no significant change in liposome size was observed when adding increasing concentrations of PEG-lipids during liposome synthesis by HFF.

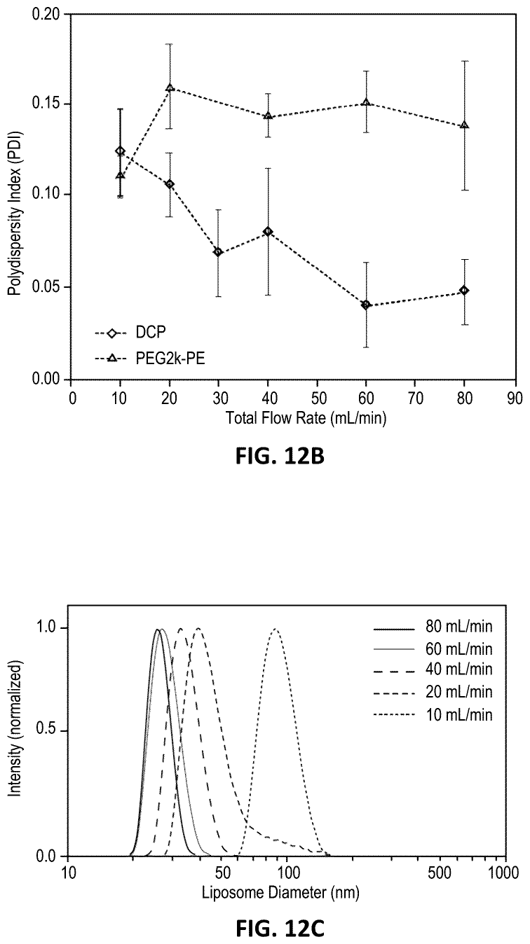

To investigate this issue for the MVF technique, a high concentration (10 mol %) of PE conjugated to PEG-2000 was introduced to the lipid feed solution before operating the hydrocyclonic flow cell at FRR=50 and 10 mM lipid concentration while varying the total flow rate. The resulting measurements of vesicle size are shown in FIG. 12A, with corresponding polydispersity shown in FIG. 12B. The presence of PEGylated lipids during vesicle formation reduced vesicle size at all flow rates, with a maximum decrease of nearly 60% at a total flow rate of 80 mL/min, leading to a minimum mean vesicle diameter of 27 nm. However, polydispersity for the PEG-lipid mixture remained nearly invariant with flow rate, with an average PDI of 0.14 over the tested range.

One of the advantages of the disclosed MVF technique is the ability to generate size-controlled liposomes by taking advantage of simultaneous flow focusing and vortical mixing, while operating at bulk flow rates that can be significantly higher than conventional microfluidic techniques, such as HFF or chaotic advection micromixer platforms. Maximum flow rates can be dictated by the need for laminar flow conditions within the flow cell to maintain efficient focusing. Based on the main chamber radius of the hydrocyclonic flow cell, the Reynolds number was found to approach the laminar limit when operating at a total flow rate of 60 mL/min, and begins to enter the transitional regime at the highest tested flow rate of 80 mL/min. Higher flow rates were thus avoided to improve throughput without inducing turbulent flow, which could otherwise destabilize the focusing zone and lead to higher polydispersity and an overall reduction in size control.

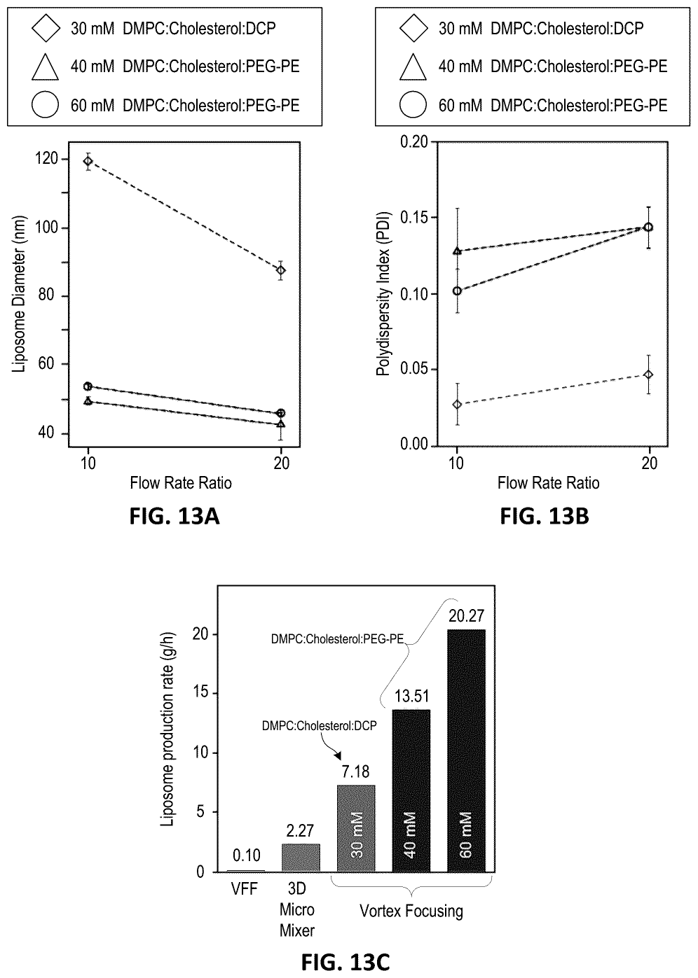

However, increasing the lipid feed concentration represents an alternative path to higher mass production rates independent of lipid and buffer flow conditions. To explore this alternative, liposomes were synthesized for each lipid mixture with concentrations approaching the lipid solubility limit while operating at the maximum flow rate of 80 mL/min. For the DCP-based lipid mixture, solubility in dehydrated ethanol was maintained for lipid concentrations up to 30 mM, while the PEGylated lipid mixture remained soluble up to 60 mM. The results of the these tests are shown in FIGS. 13A-13C. As shown in FIG. 13C, mass production rates as high as 7.2 g/h were achieved for the DCP-based liposomes, while the higher solubility limit of the PEGylated lipid mixture enabled a maximum rate over 20 g/h. This throughput is more than 50 times higher than that previously demonstrated for high aspect ratio HFF, and nearly an order of magnitude higher than emerging chaotic advection mixing platforms developed for high throughput operation. For all tested flow rates, the full incorporation of lipids into the desired bilayer vesicles was inferred from the lack of peaks associated with smaller micelles or larger lipid aggregates in the resulting light scattering data.

The MVF technique thus combines the advantages of HFF and chaotic advection mixing to enable size-tunable liposome generation while operating at high levels of lipid flux. Moreover, unlike conventional microfluidic techniques, the MVF technique offers production rates compatible with manufacturing scale liposomal drug production lines, e.g., in a range of 1-60 g/h, while also providing greater control over both mean size and size variance for the resulting vesicles, particularly while operating at higher lipid mass transport rates. Nanoparticle systems employing the MVF technique can be operated as continuous-flow setup with minimal labor and infrastructure, while avoiding the sequential processing steps associated with conventional batch methods.

### CONCLUSION

Any of the features illustrated or described herein, for example, with respect to FIGS. 1A-13C, can be combined with any other feature illustrated or described herein, for example, with respect to FIGS. 1A-13C to provide materials, systems, devices, structures, methods, and embodiments not otherwise illustrated or specifically described herein. All features described herein are independent of one another and, except where structurally impossible, can be used in combination with any other feature described herein. In view of the many possible embodiments to which the principles of the disclosed technology may be applied, it should be recognized that the illustrated embodiments are only examples and should not be taken as limiting the scope of the disclosed technology. Rather, the scope is defined by the following claims. We therefore claim all that comes within the scope and spirit of these claims.

1. A method for forming a plurality of nanoparticles, the method comprising:
(a) directing a first inlet flow along an axial direction in a hydrocyclonic flow cell, the first inlet flow comprising first constituent molecules; and
(b) at a same time as (a), directing one or more second inlet flows along a circumferential direction of the hydrocyclonic flow cell, each second inlet flow comprising a buffer solution,
wherein the directing of (a) and (b) are such that the first inlet flow is subjected to flow focusing by a surrounding primary vortex formed by the one or more second inlet flows, so as to generate a flow comprising the plurality of nanoparticles at an outlet of the hydrocyclonic flow cell, and
each nanoparticle is formed by a respective plurality of the first constituent molecules.

2. The method of claim 1, wherein:
the hydrocyclonic flow cell comprises:
a cylindrical portion comprising a circumferentially-extending wall;
an inlet nozzle surrounded by the circumferentially-extending wall of the cylindrical portion and extending toward the outlet; and
a conical portion that extends from an axial end of the circumferentially-extending wall of the cylindrical portion toward the outlet, the conical portion being tapered along at least a portion of its length along the axial direction;
the first inlet flow is directed into the hydrocyclonic flow cell via the inlet nozzle;
each second inlet flow is directed into the cylindrical portion; and
the inlet nozzle and the outlet are substantially co-axial.

3. The method of claim 2, wherein a radially-outer surface of the inlet nozzle is tapered along at least a portion of its length along the axial direction.

4. The method of claim 1, further comprising:
(c) at a same time as (a), directing one or more third inlet flows in the hydrocyclonic flow cell, each third inlet flow being adjacent to the first inlet flow along a radial direction of the hydrocyclonic flow cell and comprising second constituent molecules,
wherein the directing of (a), (b), and (c) is such that each nanoparticle comprises the respective plurality of the first constituent molecules and one or more of the second constituent molecules.

5. The method of claim 4, wherein the second constituent molecules comprise a drug.

6. The method of claim 4, wherein the one or more third inlet flows comprises an annular flow substantially centered with and surrounding the first inlet flow.

7. The method of claim 1, wherein:
the first inlet flow comprises second constituent molecules, and
the directing of (a) and (b) are such that each nanoparticle comprises the respective plurality of the first constituent molecules and one or more of the second constituent molecules.

8. The method of claim 7, wherein the second constituent molecules comprises a drug.

9. The method of claim 1, wherein:
the one or more second inlet flows comprise second constituent molecules, and
the directing of (a) and (b) are such that each nanoparticle comprises the respective plurality of the first constituent molecules and one or more of the second constituent molecules.

10. The method of claim 9, wherein the second constituent molecules comprises a drug.

11. The method of claim 1, wherein the first constituent molecules comprise an organic material or an inorganic material.

12. The method of claim 1, wherein the first constituent molecules comprise lipids, and each nanoparticle is a liposome or lipid nanoparticle.

13. The method of claim 12, wherein at least some of the lipids comprise polyethylene glycol (PEG).

14. The method of claim 1, wherein a flow rate of the first inlet flow and/or a flow rate of the one or more second inlet flows are selected so as to provide predetermined sizes for the plurality of nanoparticles.

15. The method of claim 1, wherein the directing of (a) and (b) are such that the plurality of nanoparticles are generated at a rate of at least 20 g/h.

16. The method of claim 1, wherein the generated plurality of nanoparticles have diameters less than or equal to 100 nm and a polydispersity index less than or equal to 0.2.

17. A nanoparticle synthesis system comprising:
a hydrocyclonic flow cell comprising:
a cylindrical section comprising a circumferentially-extending wall with one or more tangentially-oriented inlet ports;
an inlet nozzle surrounded by the circumferentially-extending wall of the cylindrical section and providing an axially-oriented inlet port; and
a conical section that extends from an axial end of the circumferentially-extending wall of the cylindrical section toward an axially-oriented outlet port, the conical section being tapered along at least a portion of its length along an axial direction,
wherein the axially-oriented inlet port and the axially-oriented outlet port are substantially co-axial, and
a radially-outer surface of the inlet nozzle is tapered along at least a portion of its length along the axial direction.

18. The nanoparticle synthesis system of claim 17, wherein:
the hydrocyclonic flow cell further comprises a second inlet nozzle surrounded by the circumferentially-extending wall of the cylindrical section; and
the second inlet nozzle provides an annular port proximal to and substantially centered with the axially-oriented inlet port of the inlet nozzle along a radial direction.

19. The nanoparticle synthesis system of claim 17, wherein:
(a) a diameter of the axially-oriented inlet port of the inlet nozzle is less than or equal to 500 μm;
(b) a diameter of the axially-oriented outlet port is less than or equal to 500 μm;
(c) a thickness of a wall of the inlet nozzle that defines the axially-oriented inlet port is less than 150 μm along a radial direction; or
any combination of (a)-(c).

20. The nanoparticle synthesis system of claim 17, further comprising:
a reservoir of buffer solution fluidically coupled to the one or more tangentially-oriented inlet ports; and
a reservoir of lipid solution fluidically coupled to the axially-oriented inlet port of the inlet nozzle.
